(19) United States
(12) Patent Application Publication
(10) Pub. No.: US 2013/0261382 A1
(43) Pub. Date: Oct. 3, 2013

(54) Devices and Methods for the Treatment of Obesity

(71) Applicant: Pablo ACOSTA, Newark, CA (US)

(72) Inventor: Pablo ACOSTA, Newark, CA (US)

(73) Assignee: VIBRYNT, INC., Redwood Drive, CA (US)

(21) Appl. No.: 13/437,656

(22) Filed: Apr. 2, 2012

(51) Int. Cl. A61B 17/122

(52) U.S. Cl. 600/37

(57) ABSTRACT

A device includes a reinforcing member with a cross-section that defines a channel. The channel is configured to reinforce a wound. The device further includes a constraining body attachable to the reinforcing member and the constraining body is configured to constrain a patient's stomach. Methods for implanting the device and treating a patient are provided.

### BACKGROUND OF THE INVENTION

Embodiments of the present invention relate generally to medical devices and methods and more particularly to minimally invasive devices, systems and methods for treating obesity.

Obesity has become a major health concern, both nationally and internationally. The National Center for Health Statistics (NCHS) estimates that over 120 million Americans are overweight, including about 56% of the adult population. Of these, about 52 million are considered obese, as measured by a body mass index (BMI) of 30% or greater. In Europe, an estimated 77 million people are obese, as measured by the same standard. This problem is not limited to western nations, as many developing countries are reported to have obesity rates over 75% of the adult population.

Co-morbidities that are associated with obesity include, but are not limited to type II Diabetes, high blood pressure, sleep apnea, stroke and arthritis, the symptoms of which often tend to be lessened or alleviated upon loss of weight by a person so affected.

In the U.S., options for treatment of obesity are currently quite limited. Current treatment methodologies typically rely upon surgically introducing a “malabsorptive” environment in the gastro-intestinal tract, a restrictive environment, or a combination of these. One available treatment method is gastric bypass surgery and another is referred to as gastric banding (one of these techniques if referred to as the LAPBAND™ procedure). These procedures are limited to only those patients with a BMI over 40 (or over 35, with co-morbidities present).

Gastric bypass procedures incur a great deal of morbidity and create a malabsorptive state in the patient by bypassing a large portion of the intestines. Serious side effects, such as liver failure have been associated with this procedure, as well as chronic diarrhea. Another surgical procedure that has a high degree of morbidity associated with it is known as the “Gastric Bypass Roux-en-Y” procedure. This procedure reduces the capacity of the stomach by creating a smaller stomach pouch. The small space holds only about one ounce of fluid. A tiny stomach outlet is also surgically created to slow the speed at which food leaves the stomach. Staples are used to create a small (15 to 20 cc) stomach pouch, with the rest of the stomach being stapled completely shut and divided from the stomach pouch. The small intestine is divided just beyond the duodenum, brought up, and connected to the newly formed stomach pouch. In addition to the considerable morbidity associated with this procedure, other disadvantages include “dumping syndrome”, where stomach contents are literally “dumped” rapidly into the small intestine which may lead to nausea, weakness, sweating, faintness, and diarrhea; hernias resulting from the surgery; gallstones; leakage of the connection between the pouch and the intestine; stretching of the pouch that was formed; nutritional deficiencies; and possible dehiscence of the staples.

The LAPBAND™ is a band that, when placed, encircles the fundus-cardia junction and is inflatable to constrict the same. It does not reduce the volume of the stomach, but rather restricts passage of food into the stomach, the theory being that the patient will feel satiety with a much less volume of food than previously. Although the LAPBAND™ procedure is less invasive than a gastric bypass procedure, it also typically achieves less weight loss. Further, it is not a simple procedure and requires a substantial amount of training by a surgeon to become proficient in performing the procedure. Also, a substantial amount of dissecting and suturing is required because the pathway by which the band is introduced is not an existing pathway, and must be established by dissection. Great care is required to avoid blood vessels and nerves that may be in the intended pathway to be created by the dissection. After placing the band around the fundus-cardia junction, the ends of the band must be connected together and then it must be cinched down into place. Additionally, complications such as erosion at the fundus-cardia junction, slippage of the band from its intended location, nausea/vomiting, gastroesophageal reflux, dysphagia and lack of effectiveness in causing weight loss have been reported.

Gastrointestinal sleeves have been implanted to line the stomach and/or a portion of the small intestines to reduce the absorptive capabilities of the small intestine and/or to reduce the volume in the stomach, by reducing the available volume to the tubular structure of the graft running therethrough. Although weight loss may be effective while these types of devices are properly functioning, there are complications with anchoring the device within the stomach/GI tract, as the stomach and GI tract function to break down things that enter into them and to move/transport them through. Accordingly, the integrity of the anchoring of the device, as well as the device itself may be compromised over time by the acids and actions of the stomach and GI tract.

A sleeve gastrectomy is an operation in which the left side of the stomach is surgically removed. This results in a much-reduced stomach that is substantially tubular and may take on the shape of a banana. This procedure is associated with a high degree of morbidity, as a large portion of the stomach is surgically removed. Additionally, there are risks of complications such as dehiscence of the staple line where the staples are installed to close the surgical incisions where the portion of the stomach was removed. Further, the procedure is not reversible.

In the laparoscopic duodenal switch, the size of the stomach is reduced in similar manner to that performed in a sleeve gastrectomy. Additionally, approximately half of the small intestine is bypassed and the stomach is reconnected to the shortened small intestine. This procedure suffers from the same complications as the sleeve gastrectomy, and even greater morbidity is associated with this procedure due to the additional intestinal bypass that needs to be performed. Still further, complications associated with malabsorption may also present themselves.

Although procedures to surgically reduce stomach volume can produce weight loss, in some patients the weight loss is not permanent. For example, in the case of vertical sleeve gastrectomies, the sleeve expands over time due to the patient overeating and at least some of the reduction in stomach volume is lost. Other procedures can have similar outcomes where the reduction in volume is lost due to patient overeating or partial failure of the device.

U.S. Patent Publication No. 2005/0261712 to Balbierz et al. describes capturing a device against the outer surface of the stomach wall to form a restriction that appears to function similarly to the restriction imposed by the LAPBAND™. The anchoring of the devices disclosed relies upon placement of features against the internal wall of the stomach to form an interlock with the device that is placed against the external wall of the stomach.

U.S. Patent Publication No. 2005/0267533 to Gertner discloses devices for treatment of obesity that use one or more anchoring mechanisms that are passed through the wall of the stomach to establish an anchor. The stomach is reduced in size by passing the devices through the stomach wall on opposite sides of the stomach and compressing the walls together to eliminate a portion of the interior space within the stomach. Gertner also discloses an embodiment in which an extra-gastric balloon is placed anteriorly of the stomach and attached to the abdominal wall using one of the anchoring mechanisms described.

U.S. Pat. No. 6,981,978 to Gannoe discloses devices for reducing the internal cavity of the stomach to a much smaller volume, which may be used to carry out a bypass procedure. Stapling is employed to isolate the smaller volume in the stomach, and thus the same potential disadvantages are present as with other stapling procedures described herein.

U.S. Pat. No. 6,186,149 to Pacella et al. describes an occluder device that can be used as a dietary control device (see FIG. 8C). The occluder device is placed against the wall of the stomach and inflated to press inwardly on the stomach wall. A frame is wrapped around the stomach wall and is inflated to press against the stomach wall. However, there is no disclosure of how the frame might be adjusted to maintain a position relative to the stomach wall as the size of the stomach varies.

The risk and invasiveness factors of currently available surgeries are often too great for a patient to accept to undergo surgical treatment for his/her obesity. Accordingly, there is a need for less invasive, yet effective surgical treatment procedures for morbidly obese patients (patients having a BMI of 35 or greater). Also, since the current surgical procedures are currently indicated only for those patients having a BMI of 40 or greater, or 35 or greater when co-morbidities are present, it would be desirable to provide a surgical procedure that would be available for slightly less obese patients, e.g., patients having a BMI of 30 to 35 who are not indicated for the currently available surgical procedures. It would further be desirable to provide a surgical procedure that would be indicated for obese patients having a BMI in the range of 30-35, as well as for more obese patients.

There is a need for devices and methods for treating obesity with minimally invasive devices that substantially preserve the reduced volume of a stomach following a bariatric procedure and optionally provide an adjustable, constrictive force on the stomach.

### BRIEF SUMMARY OF THE INVENTION

Certain embodiments of the present invention are related to a device for the treatment of obesity. The device includes a reinforcing member with a cross-section that defines a channel. The channel is configured to reinforce a wound. The device further includes a constraining body attachable to the reinforcing member. The constraining body is configured to constrain a patient's stomach. In certain embodiments, the reinforcing member includes articulated segments. In certain embodiments, the reinforcing member includes pinching segments. In certain embodiments, the constraining body includes at least one inflatable bladder.

Certain embodiments of the present invention are related to a method for the treatment of obesity. The method is preferably performed as a laparoscopic procedure but it is within the scope of the invention for the method to be performed as an open surgical procedure or a trans-oral procedure. The method includes accessing an extra-gastric abdominal space of a patient and introducing a device into the extra-gastric abdominal space. The device includes a reinforcing member and a constraining body. The method further includes positioning the reinforcing member about a wound on the stomach of the patient and positioning the constraining member about at least a portion of the stomach of the patient. In certain embodiments, method further includes applying pressure on the closed wound via the reinforcing member. In certain embodiments, the method further includes applying a sealant material within the channel of the reinforcing member. The method further includes fastening at least a portion of the constraining member to another portion of the constraining member. In certain embodiments, the method further includes at least partially inflating at least one inflatable bladder included with the constraining body.

### BRIEF DESCRIPTION OF SEVERAL VIEWS OF THE DRAWINGS

FIG. 1A illustrates a stomach of a patient after undergoing a vertical banded gastroplasty procedure to reduce stomach volume.

FIG. 1B illustrates a stomach of a patient after undergoing a vertical sleeve gastrectomy procedure to reduce stomach volume.

FIG. 1C illustrates a cross-sectional view of a stomach of a patient after undergoing a vertical sleeve gastrectomy procedure to reduce stomach volume.

FIGS. 2A, 2B, and 2C illustrate perspective views of a reinforcing member according to certain embodiments of the invention.

FIG. 3 illustrates a cross-sectional view of a stomach of a patient after undergoing a vertical sleeve gastrectomy procedure to reduce stomach volume and a reinforcing member according to certain embodiments of the invention.

FIGS. 4A, 4B, and 4C illustrate cross-sectional views of reinforcing members according to certain embodiments of the invention.

FIG. 5 illustrates a perspective view of articulated segments of a reinforcing member according to certain embodiments of the invention.

FIG. 6A illustrates a plan view of the interior (stomach-facing) surface of a constraining body according to certain embodiments of the invention.

FIG. 6B illustrates a plan view of the exterior surface of a constraining body according to certain embodiments of the invention.

FIG. 7 illustrates a plan view of the interior (stomach-facing) surface of a constraining body according to certain embodiments of the invention.

FIG. 8 illustrates a cross-sectional view of a constraining body according to certain embodiments of the invention.

FIG. 9 illustrates a cross-sectional view of a stomach of a patient after undergoing a vertical sleeve gastrectomy procedure to reduce stomach volume, a reinforcing member, and a constraining body attached to the reinforcing member according to certain embodiments of the invention.

FIG. 10 illustrates a perspective view of a constraining body having inflatable bladders according to certain embodiments of the invention.

FIG. 11 illustrates a cross-sectional view of a stomach of a patient after undergoing a vertical sleeve gastrectomy procedure to reduce stomach volume, a reinforcing member, and a constraining body attached to the reinforcing member according to certain embodiments of the invention.

### DETAILED DESCRIPTION OF THE INVENTION

Embodiments of the present invention are useful in the surgical treatment of obesity. The description, figures, and examples herein relate to devices and methods for the containment or constriction of the stomach.

Before the present devices and methods are described, it is understood that this invention is not limited to particular embodiments described, as such may, of course, vary. It is also understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

Short summaries of certain terms are presented in the description of the invention. Each term is further explained and exemplified throughout the description, figures, and examples. Any interpretation of the terms in this description should take into account the full description, figures, and examples presented herein.

The singular terms “a,” “an,” and “the” include plural referents unless the context clearly dictates otherwise. Thus, for example, reference to an object can include multiple objects unless the context clearly dictates otherwise. Similarly, references to multiple objects can include a single object unless the context clearly dictates otherwise.

The terms “substantially,” “substantial,” and the like refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

The terms “attach,” “attaching,” “attached,” “attachment” and the like refer to one part being coupled to another part in a substantially fixed manner. The two parts may be attached directly or through one or more intermediate parts provided that the intermediate parts preserve the substantially fixed manner of the coupling.

The terms “inflate,” “inflatable,” “inflated,” “inflating,” “inflation,” and the like refer to an object or region of an object becoming larger in size or volume. In certain cases herein, such inflation is accomplished by at least partially filling an internal volume of the object or region. However, it is understood that these terms include cases where an internal volume of the object or region is filled but the object or region does not stretch or grow. In such cases where filling the object does not cause the object or region to stretch or grow, the act of at least partially filling the internal volume of the object or region is the act of inflation. Conversely, the term “uninflated” and the like refer to both the condition of an object or a region prior to it becoming larger in size or volume and to the condition of an object or region prior to its internal volume being at least partially filled.

The term “channel” and the like refers to both the structure that defines a conduit with at least one open side or face and the void or negative space defined by such a structure, as the context dictates. For example, two side walls connected by a base wall can be referred to as a channel as can the void defined by these features. It is understood that the shape of a channel is not limited to u-shapes or v-shapes but can includes various other shapes both complex and simple.

The terms “reinforce,” reinforced,” “reinforcing,” and the like refer to one object supporting another to some degree. Such support includes providing a range of forces from active pressure to simple contact.

The terms “constrain,” “constrained,” “constraining,” and the like refer to both the application of active forces that apply pressure to reduce the size of an object and to the provision of a passive barrier that limits the expansion of an object to a larger size.

The term “wound” and the like refers to areas of iatrogenic trauma to tissue, including, but not limited to, incisions and punctures. It is understood that the term “wound” includes areas of recent trauma and those that have occurred during a past procedure.

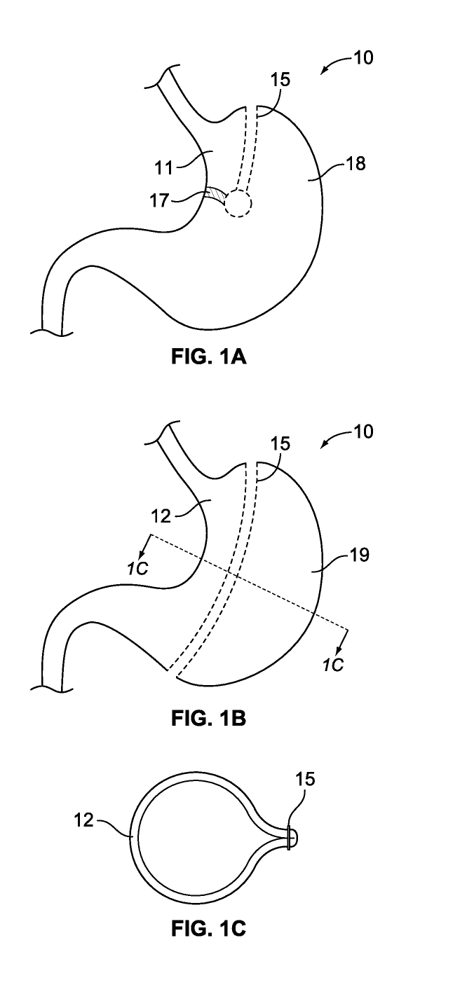

FIG. 1A illustrates a stomach of a patient after undergoing a vertical banded gastroplasty procedure to reduce stomach volume. In typical vertical banded gastroplasty procedures, forming pouch 11 reduces the volume of stomach 10. Pouch 11 is formed by placing at least one vertical row, and often two vertical rows of wound closure devices 15 such as surgical staples on the upper portion of stomach 10 near the fundus and band 17 at the inferior part of pouch 11. FIG. 1B illustrates a stomach of a patient after undergoing a vertical sleeve gastrectomy procedure to reduce stomach volume. In typical sleeve gastrectomy procedures, left side 19 of stomach 10 is removed. Right side 12 of stomach 10 remains intact as a tube-like structure bounded by a line of wound closure devices 15. FIG. 1C illustrates a cross-sectional view of a stomach of a patient after undergoing a vertical sleeve gastrectomy procedure to reduce stomach volume. FIG. 1C illustrates the reduced volume of stomach 10 and the portion of stomach 10 that is fixed by wound closure device 15. In the procedures illustrated in FIGS. 1A, 1B, and 1C, wound closure device 15 is depicted as a surgical staple, but it is understood that any wound closure device is within the scope of the invention.

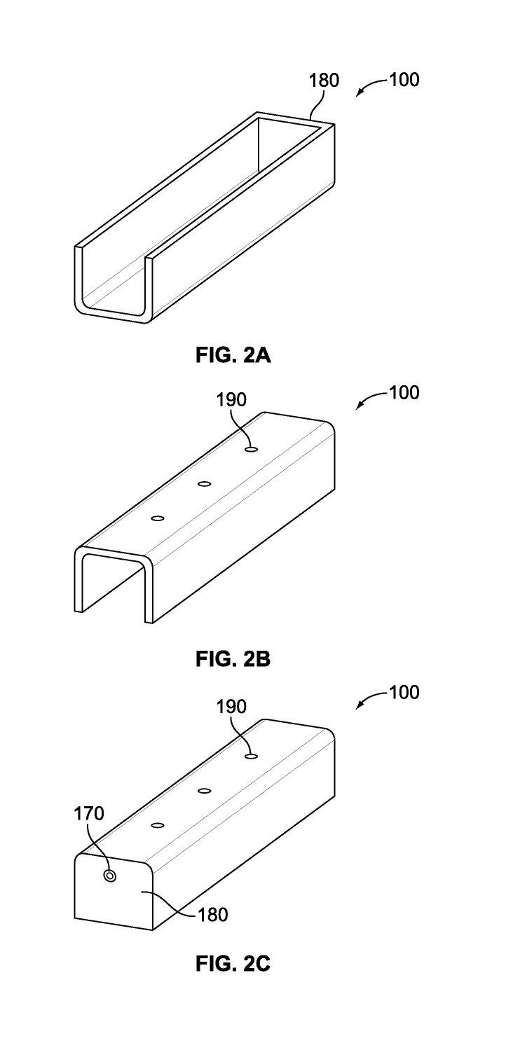

FIGS. 2A, 2B, and 2C illustrate perspective views of a reinforcing member according to certain embodiments of the invention. In certain embodiments, reinforcing member 100 is configured to have a channel capable of fitting over a wound closed with wound closure devices. Reinforcing member 100 has a channel. FIG. 2A depicts the channel of reinforcing member 100 facing upwards. Reinforcing member 100 can optionally include end cap 180 positioned at one or both ends of the channel. FIG. 2B depicts the channel of reinforcing member 100 facing downward, which reveals attachment points 190. Attachment points 190 are configured to facilitate the attachment of a constraining body to reinforcing member 100. One of the novel aspects of embodiments of the present invention is that reinforcing member 100 serves as an anchor for a constraining body that can constrain the stomach of the patient. FIG. 2C depicts the channel of reinforcing member 100 facing downward in an embodiment of reinforcing member 100 in which both ends of reinforcing member 100 includes end cap 180. FIG. 2C depicts end cap 180 as including port 170. Port 170 facilitates the introduction of a sealant material into the channel. The sealant material can fill any void left between the channel and the tissue of the wound inside the channel. The sealant material can also help seal the wound and help anchor reinforcing member 100 to stomach 10. It is understood that there may be a port on each end cap of a reinforcing member with an end cap on each end. Further, ports may be located on either side wall or the bottom of the channel of reinforcing member 100. Still further, it is understood that such ports are optional and, according to certain embodiments, sealant may be placed in the channel via the uncapped ends of reinforcing member 100 or directly within the channel prior to fitting reinforcing member 100 about the wound. Any type of surgical sealant or surgical glue can be used in conjunction with reinforcing member 100, including but not limited to, fibrin sealants, cyanoacrylates, collagen-based compounds, glutaraldehyde glues and hydrogels.

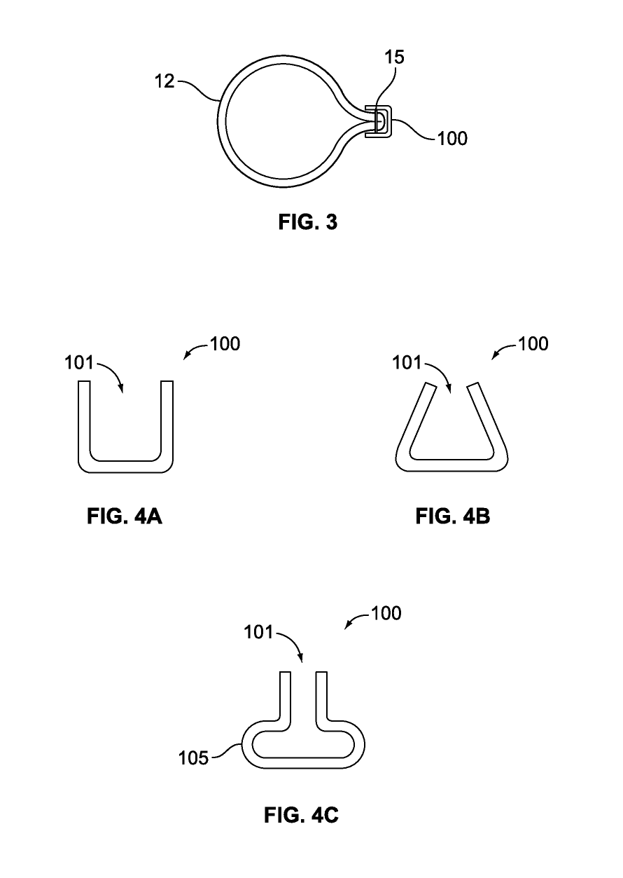

FIG. 3 illustrates a cross-sectional view of a stomach of a patient after undergoing a vertical sleeve gastrectomy procedure to reduce stomach volume and a reinforcing member according to certain embodiments of the invention. FIG. 3 depicts right side 12 of the stomach similar to the illustration in FIG. 1C. In FIG. 3, reinforcing member 100 is positioned about wound closure device 15 and the wound that device 15 closes. According to certain embodiments, a sealant material is also applied to the junction of the wound and reinforcing member 100.

The length and configuration of the reinforcing member can vary such that it reinforces just one closure device or multiple wound closure devices. The line of wound closure devices on a stomach may be fairly straight and a single reinforcing member may be able to be positioned about the majority of the wound closure device, preferably all of the wound closure devices, without placing undesired forces on the wound. Undesired forces include forces that distend the stomach or displace the line of wound closure devices in a way that increases the possibility of leakage or failure of the wound closure. In embodiments where the reinforcing member is configured to be positioned about one wound closure device or a few wound closure devices, more than one reinforcing member may be placed about the wound. In such embodiments, each reinforcing member may optionally have sealant applied to its channel via the methods described herein.

FIGS. 4A, 4B, and 4C illustrate cross-sectional views of reinforcing members according to certain embodiments of the invention. FIG. 4A depicts reinforcing member 100 with a cross-section that defines channel 101, and it is understood that the height of the side walls of channel 101 can vary from the depiction and the width of channel 101 can vary from the depiction in FIG. 4A. FIG. 4B depicts reinforcing member 100 with a cross-section in which the mouth or upper end of channel 101 is narrower than the base or lower end of channel 101. It is understood that height of the side walls, the width of the base of channel 101, the width of the mouth of channel 101, and the angle the side walls make with the base of channel 101 can all vary from the depiction in FIG. 4B. FIG. 4C depicts reinforcing member 100 with a cross-section in which the base of channel 101 includes flange 105. It is understood that height of the side walls, the width of the base of channel 101, the width of the mouth of channel 101, and width and thickness of the flange can all vary from the depiction in FIG. 4C. Flange 105 may facilitate the attachment of a constraining body as described herein.

FIGS. 4A, 4B, and 4C depicts just a few possible embodiments of the cross-section of the reinforcing member. Other embodiments of the cross-section and the channel it defines are within the scope of the invention. It is understood that regardless of the specific geometry of the cross-section of the reinforcing member, at least a portion of the channel is dimensioned and configured to be positioned about the wound on a patient's stomach following a procedure to reduce the volume of the stomach. Specifically, the width of the channel opening and the depth of the channel are configured to be positioned about such a wound.

According to certain embodiments, the reinforcing member may apply pressure to the wound. Specifically, the reinforcing member may apply a pinching type of pressure to the wound that may facilitate the wound remaining closed and free from leakage. Reinforcing members may be configured to apply a pinching force in any suitable way. For example, reinforcing member 100 of FIG. 4B may be configures such that the relatively narrower opening of channel 101 applies a pinching force. In the embodiment depicted in FIG. 4B, the side walls may be spread apart slightly while reinforcing member 100 is positioned fairly snugly about the wound so that when released the side walls would exert a pinching force on the wound. Other cross-sections may function in a similar way to apply a pinching force when positioned about the wound.

Reinforcing members may be formed from any suitable biocompatible material. Suitable materials include those that are capable of maintaining their shape and optionally applying suitable pinching force to a wound. Examples of such materials include biocompatible polymers (including but not limited to, polyteterafluoroethylene and similar fluorinated polymers, silicone rubbers, high and ultrahigh molecular weight polyethylene, and nylons), metals (including but not limited to, stainless steel, titanium, and nickel-titanium alloys), and ceramics. Reinforcing members may be formed from laminated materials or composites of materials. For example, a reinforcing member may be formed from a metal, such as a nickel-titanium alloy, that is encapsulated by a silicone rubber. The nickel-titanium alloy may be configured to supply a pinching force and the encapsulating silicone rubber may provide an atraumatic interface with the wound and surrounding tissue.

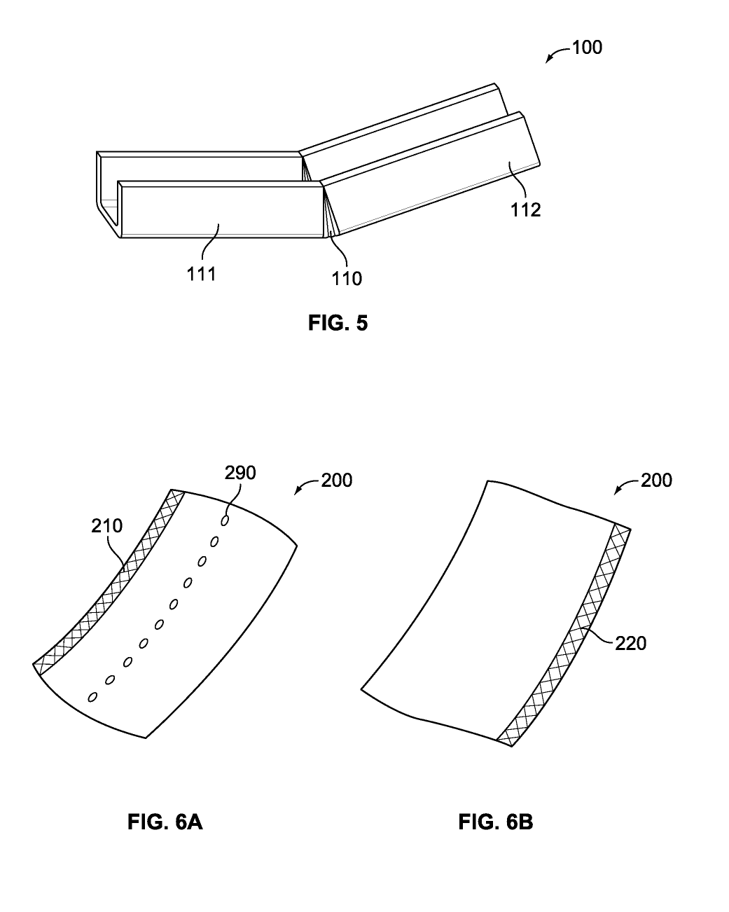

FIG. 5 illustrates a perspective view of articulated segments of a reinforcing member according to certain embodiments of the invention. Reinforcing member 100 includes segment 111 and segment 112. In between segment 111 and segment 112 is articulation 110 that allows segment 111 and segment 112 to move with respect to one another. Articulation 110 may be configured such that it allows segment 111 and segment 112 to change angle with respect to each other but maintain alignment along their respective center lines. That is, articulation 110 may be configured to allow just one axis of relative motion between segments. Alternately, articulation 110 may be configured such that it allows segment 111 and segment 112 to change angles along two axes with respect to each other. In either case, articulation 110 optionally may be configured to allow segment 111 and segment 112 to move farther apart from each other in addition to the one or two axes of angular motion. It is understood that while FIG. 5 depicts one articulation between two segments, a reinforcing member may include three or more segments with an articulation between each adjacent segment. Such reinforcing members having multiple articulated segments may be useful in positioning a reinforcing member about a wound while following the contour of the patient's stomach. Further, multiple reinforcing members having multiple articulated segments may be positioned on a single patient's stomach to provide the desired amount of coverage of the wound.

FIG. 6A illustrates a plan view of the interior (stomach-facing) surface of a constraining body according to certain embodiments of the invention. In certain embodiments, constraining body 200 is made from a flexible material. In certain embodiments, constraining body 200 is made from a flexible and stretchable material. Some example of types of materials suitable for making constraining body 200 include biocompatible fabrics, cloths, and textiles made through weaving, knitting, spreading, crocheting, bonding or other techniques, such as silicone, polyester, polyvinyl chloride, polyethylene, poly ether-ether ketone, polycarbonate, polyurethane, polypropylene, and nylon. Other examples of types of materials suitable for making constraining body 200 include biocompatible films made by conventional techniques; such films can be made from biocompatible polymers such as silicone, polyester, polyvinyl chloride, polyethylene, poly ether-ether ketone, polycarbonate, polyurethane, polypropylene, and nylon. The fabrics and films useful for making constraining body 200 may be used as a single layer or in multiple layers. Further, the fabrics and films may be configured as composites, with the material varying from layer to layer in multilayer configurations and also within each layer in both a single layer and multilayer configuration. The layers in multilayer arrangements may be attached to one another by conventional techniques including stitching, adhesive bonding, heat (or other energy) bonding, and combination thereof. The multilayer configurations may be useful in distributing forces about a relatively wide surface area of the stomach.

As depicted in FIG. 6A, constraining body 200 includes at least one attachment point 290. FIG. 6A depicts multiple attachment points 290 and depicts them arranged in a row extended longitudinally about the rough center axis constraining body 200. It is understood that attachment point 290 may be arranged in a variety of other ways and are not limited to any of the aspects of the arrangement (e.g., linear, about the center axis, and extending from one edge to the other) depicted in FIG. 6A. Attachment points 290 are configured to attach with attachment points 190 of the various embodiments of reinforcing member 100. Preferably, the specific arrangement of attachment points 290 conforms to the positioning of attachment points 190 on the one or more reinforcing members 100 positioned about the wound on the stomach. In certain embodiments, attachment points 290 are capable of being moved and configured by the user to facilitate the attachment process with the one or more reinforcing members. For example, in an embodiment in which constraining body 200 is made from a fabric, attachment points 290 may be moved to various places on constraining body 200 without adversely affecting the functional properties of constraining body 200.

FIG. 6A depicts constraining body 200 including first fastening region 210. First fastening region 210 includes one or more fastening devices. FIG. 6B illustrates a plan view of the exterior surface of a constraining body according to certain embodiments of the invention. FIG. 6B depicts constraining body 200 including second fastening region 220. Second fastening region 210 includes one or more fastening devices. First fastening region 210 and second fastening region 220 are configured to fasten to one another when constraining body 200 is positioned about the stomach of the patient.

Some examples of suitable fastening devices for one or both such fastening regions include but are not limited to holes, grommets, loops, hooks, snaps, ties, knots, needles, pins, threaded bolts or screws, threaded nuts, clips, magnets, and the like. In certain embodiments of the invention, such as hook and loop, snaps, bolts and nuts, or similar paired fastening devices, it is necessary that both parts being fastened have a complementary fastening device. For example, first fastening region 210 may have a male part of a snap to be attached to a corresponding female part of a snap included in second fastening region 220. In certain embodiments, the fastening devices are varied in one or both fastening regions such that a single fastening region may have different types of coupling points.

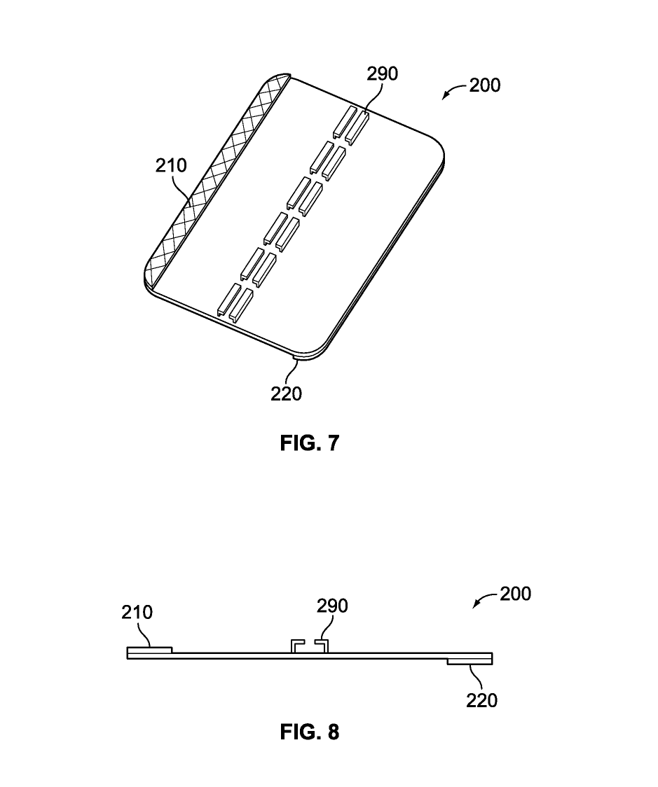

FIG. 7 illustrates a plan view of the interior (stomach-facing) surface of a constraining body according to certain embodiments of the invention. Constraining body 200 includes first fastening region 210 and attachment points 290. FIG. 8 illustrates a cross-sectional view of constraining body 200 according to certain embodiments of the invention. As depicted in FIG. 8, first fastening region 210 is on the interior (stomach-facing) side of constraining body 200 and second fastening region 220 is on the exterior side of constraining body 200. FIGS. 7 and 8 depict attachment points 290 as dimensioned and configured to attach to flanged reinforcement members such as the kind depicted in FIG. 4C. FIG. 7 depicts multiple attachment points 290 that could be slid onto flanged reinforcing members to anchor constraining device 200 on the patient's stomach.

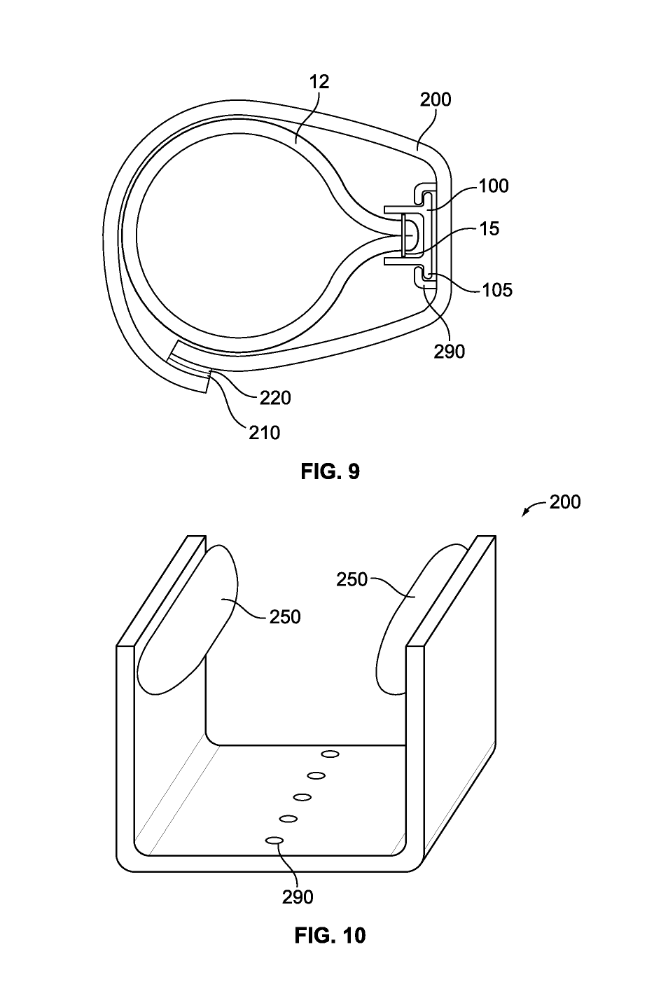

FIG. 9 illustrates a cross-sectional view of a stomach of a patient after undergoing a vertical sleeve gastrectomy procedure to reduce stomach volume, a reinforcing member, and a constraining body attached to the reinforcing member according to certain embodiments of the invention. FIG. 9 depicts right side 12 of the stomach of a patient after a vertical sleeve gastrectomy procedure similar to procedures depicted in FIGS. 1B and 1C. FIG. 9 depicts reinforcing member 100 positioned about wound closure device 15 and the stomach tissue of the wound closed by wound closure device 15. Although not pictured, it is understood that a sealant material may be applied to the junction of reinforcing member 100 and wound closure device 15 and the stomach tissue of the wound closed by wound closure device 15. Such a sealant may fill the entire void, if any, between reinforcing member 100 and wound closure device 15 and the stomach tissue of the wound closed by wound closure device 15.

FIG. 9 depicts constraining body 200 attached to reinforcing member 100 via attachment points 290. In the embodiment depicted in FIG. 9, reinforcing member 100 includes flanges 105 and attachment points 290 are dimensioned and configured to slide along flanges 105 to facilitate the attachment of constraining body 200 to reinforcing member 100. It is understood that the particular dimensions and configurations of attachment points 290 and flanges 105 depicted in FIG. 9 are exemplary only and many suitable configurations and dimensions are within the scope of the invention.

FIG. 9 depicts constraining body 200 as positioned around right side 12 of the patient's stomach. In the embodiment depicted in FIG. 9, constraining body 200 includes first fastening region 210 and second fastening region 220, and these fastening regions are fastened together to securely position constraining body 200 about right side 12 of the patient's stomach. As described herein, these fastening regions may include arrays of fastener pairs, such as the male section of a snap on first fastening region 210 and the female section of a snap on second fastening region 220. Further, these fastening regions may include multiple arrays of such fastening pairs such that the user can adjust the fit of constraining body 200 about right side 12 of the patient's stomach. As described herein with respect to the attachment points of certain embodiments, the fastening regions of constraining body 200 may be, in some embodiments, moveable and configurable by the user to provide a customizable fit of constraining body 200 about right side 12 of the patient's stomach. For example, in an embodiment in which constraining body 200 is made from a fabric, the fasteners in these fastening regions may be moved to various places on constraining body 200 without adversely affecting the functional properties of constraining body 200. Alternatively, first fastening region 210 and second fastening region 220 may each include strips of complementary fastening devices like hook-and-loop fasteners in which the width of these strips is sufficient to enable the user to provide a customizable fit of constraining body 200 about right side 12 of the patient's stomach. It is understood that the specific dimensions and configurations of the interaction between constraining body 200 and right side 12 of patient's stomach depicted in FIG. 9 are only exemplary and other dimensions and configurations are within the scope of the invention. For example, FIG. 9 depicts constraining body 200 as positioned to provide a barrier that limits the expansion of right side 12 of the patient's stomach, but constraining body 200 could be dimensioned and configured to apply constricting forces to reduce the size of right side 12 of the patient's stomach. It is understood that the fastening regions may be located at any place on the constraining body, such as in a location where the fastening regions align with the attachment points when the device is positioned about the stomach.

FIG. 10 illustrates a perspective view of a constraining body having inflatable bladders according to certain embodiments of the invention. FIG. 10 depicts an embodiment in which constraining body 200 is non-compliant such that it maintains its shape when placed about the stomach. FIG. 10 depicts a generally u-shaped constraining body 200, but it is understood that constraining body 200 may have other configurations. In embodiments where constraining body 200 is non-compliant, constraining body 200 is dimensioned and configured to directly or indirectly (e.g., via the use of inflatable bladders as described herein) apply constraining forces to the reduced volume portion of the stomach of a patient. Constraining body 200 includes attachment points 290 that, as described herein, facilitate the attachment of constraining body 200 to reinforcing member 100. Constraining body 200 including inflatable bladders 250 which may be used to apply forces to the reduced volume portion of the stomach of a patient. Inflatable bladders 250 may be coupled to any part of the interior (stomach-facing) surface of constraining body 200. It is understood that although FIG. 10 depicts two inflatable bladders 250, constraining body 200 may include more or fewer inflatable bladders. Further, although FIG. 10 depicts inflatable bladders 250 as included on non-compliant constraining body 200, it is understood that one or more inflatable bladders may be included on other embodiments of constraining body 200 that are compliant or semi-compliant.

According to certain embodiments, constraining body 200 includes selectively inflatable bladders 250 in which each bladder can be individually inflated to provide a customizable distribution of constraining or constricting forces on the reduced volume portion of the stomach of a patient. FIG. 10 depicts generally elliptically-shaped inflatable bladders 250 running roughly parallel to the longitudinal axis of constraining body 200 but it is understood that the inflatable bladders may be any shape and may be arranged other ways. Further, inflatable bladders 250 may be integrated within constraining body 200.

According to embodiments of the invention, inflatable bladders can be inflated via inflation ports that may be located at any place on the inflatable bladders or on the constraining body that allows the inflation ports to be in fluid communication with the inflatable bladders. Multiple inflatable bladders may be in fluid communication with a single inflation port such that multiple inflatable bladders may be inflated via a single inflation port. In such embodiments where a single inflation port is in fluid communication with multiple inflatable bladders, valves may allow for the selective and independent inflation of the inflatable bladders. Alternately, inflatable bladders may each have their own inflation port. One advantage of embodiments of the invention having inflatable bladders is that the shape and constrictive forces of the constraining body may be adjusted over time and repeatedly using minimally invasive inflation techniques known in the art.

Inflation bladders can be formed using compliant, semi-compliant, and/or non-compliant materials according to conventional techniques. Examples of compliant materials suitable for use in an inflatable bladder as described herein include, but are not limited to: silicone, latex rubber, and polyurethane. Examples of semi-compliant materials include, but are not limited to: nylon, polyethylene, polyester, polyamide and polyurethane. Polyurethane, nylon, polyethylene and polyester can be compliant or semi-compliant materials, depending upon the specific formulation and hardness or durometer of the material as produced. Examples of noncompliant materials that can be used in the construction of inflatable members described herein include, but are not limited to: polyethylene terepthalate (PET) and urethane. Urethane can be a compliant, semi-compliant or non-compliant material depending upon its specific formulation and hardness or durometer. Compliant, semi-compliant and noncompliant categories are not solely material limited, but are better defined by their expansion characteristics. Some materials are best suited for use in one of these categories (e.g., silicone and latex work well to make compliant structures), but other materials can be formulated and/or constructed to provide compliant, semi-compliant or noncompliant properties.

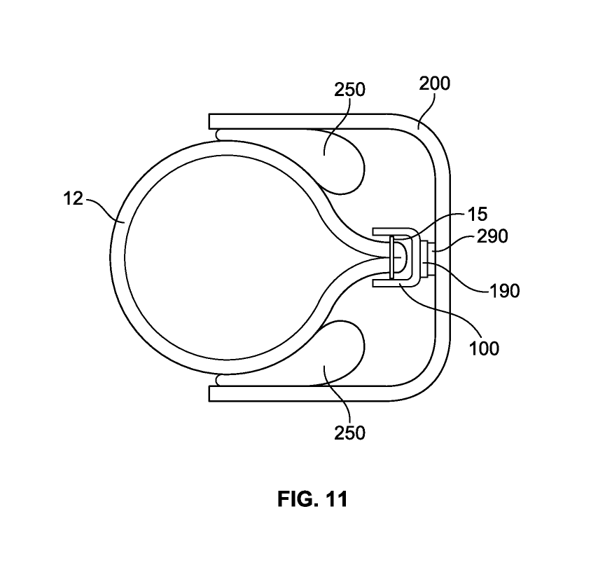

FIG. 11 illustrates a cross-sectional view of a stomach of a patient after undergoing a vertical sleeve gastrectomy procedure to reduce stomach volume, a reinforcing member, and a constraining body attached to the reinforcing member according to certain embodiments of the invention. FIG. 11 depicts non-compliant constraining body 200 positioned about right side 12 of a patient's stomach. FIG. 11 depicts inflatable bladders 250 as occupying some of the space between constraining body 200 and right side 12 of a patient's stomach. In such embodiments, inflatable bladders 250 help form a barrier to limit the expansion of right side 12 of a patient's stomach. In other embodiments, inflatable bladders may impinge upon the volume of right side 12 of a patient's stomach and constrict right side 12 of a patient's stomach such that the volume of right side 12 of a patient's stomach is reduced.

In some embodiments of the invention, reinforcing members facilitate the attachment of other extra-gastric devices to the reduced volume portion of the stomach of a patient. For example, extra-gastric balloons could be fitted with attachment points that are capable of attaching to the attachment points on one or more reinforcing members. In another example, extra-gastric stimulation devices could be fitted with attachment points that are capable of attaching to the attachment points on one or more reinforcing members.

In some embodiments, one or more reinforcing members are positioned about the wound during an initial procedure to reduce the volume of a patient's stomach, but no constraining body is attached to the one or more reinforcing members. In these embodiments, a constraining body may be placed about the reduced volume portion of a patient's stomach in a later procedure. A constraining body, or any other extra-gastric device, may be introduced to the patient's abdominal space via conventional surgical techniques or via minimally invasive surgical techniques. Since the reinforcing members and their attachment points are already positioned about the wound, the constraining body can be more easily anchored to the reduced volume portion of the patient's stomach.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A device for the treatment of obesity, comprising:
a reinforcing member wherein at least a portion of the cross-section of reinforcing member defines a channel which is configured to reinforce a wound; and
a constraining body wherein the constraining body is configured to constrain a patient's stomach.

2. The device of claim 1, wherein the reinforcing member comprises articulated segments.

3. The device of claim 1, wherein the reinforcing member comprises pinching segments.

4. The device of claim 1, wherein the reinforcing member comprises at least one port.

5. The device of claim 1, wherein the constraining body comprises a sheet configured to wrap about the patient's stomach.

6. The device of claim 1, wherein the constraining body further comprises at least one fastener.

7. The device of claim 1, wherein the constraining body comprises at least one inflatable bladder.

8. The device of claim 1, wherein the constraining body comprises a non-compliant backing member.

9. A method for the treatment of obesity, the method comprising:
accessing an extra-gastric abdominal space of a patient;
introducing a device into the extra-gastric abdominal space, the device comprising a reinforcing member and a constraining body;
positioning the reinforcing member about a wound on the stomach of the patient; and
positioning the constraining member about at least a portion of the stomach of the patient.

10. The method of claim 9 further comprising applying pressure on the closed wound via the reinforcing member.

11. The method of claim 9, wherein the reinforcing member comprises a channel.

12. The method of claim 11 further comprising applying a sealant material within the channel of the reinforcing member.

13. The method of claim 9 further comprising fastening at least a portion of the constraining member to another portion of the constraining member.

14. The method of claim 9, wherein the constraining member further comprises at least one inflatable bladder.

15. The method of claim 14 further comprising at least partially inflating the at least one inflatable bladder.

16. A method for the treatment of obesity, the method comprising:
accessing an extra-gastric abdominal space of a patient;
performing surgical procedure to reduce the volume of the stomach of the patient wherein the procedure comprises applying at least one wound closure device to the stomach;
positioning a reinforcing member about the at least one wound closure device; and
positioning the constraining member about at least a portion of the stomach of the patient.

17. The method of claim 16 further comprising attaching the constraining member to the reinforcing member.

18. The method of claim 16 wherein the at least one wound closure device is a surgical staple.

19. The method of claim 16 wherein the reinforcing member comprises articulated segments.

20. The method of claim 19 wherein positioning the reinforcing member further comprises manipulating the articulated segments to follow the contour of the stomach of the patient.
